(19) United States
(12) Patent Application Publication
(10) Pub. No.: US 2026/0083360 A1
(43) Pub. Date: Mar. 26, 2026

(54) SIGNAL PROCESSING FOR WEARABLE PHOTOGRAPHICALLY INTERROGATED COLORIMETRIC METABOLIC SENSOR

(71) Applicant: Cardiac Pacemakers, Inc., St Paul, MN (US)

(72) Inventors: Yingbo Li, Woodbury, MN (US); Michael J. Kane, St Paul, MN (US); Cullen Holten, Arden Hills, MN (US)

(21) Appl. No.: 19/338,484

(22) Filed: Sep. 24, 2025

Related U.S. Application Data
(60) Provisional application No. 63/698,681, filed on Sep. 25, 2024.

(51) Int. Cl. A61B 5/145; A61B 5/00; A61B 5/1459

(52) U.S. Cl. CPC A61B 5/14539; A61B 5/14514; A61B 5/1459; A61B 5/6833; A61B 5/685; A61B 5/7246; A61B 2562/0233

(57) ABSTRACT

Systems, methods, and devices utilize chemical sensors and include approaches for detecting a chemical indicator of a wearable device in a digital image, detecting a first color reference in the digital image, and determining a corrected color of the chemical indicator based, at least in part, on the first color reference.

### CROSS-REFERENCE TO RELATED APPLICATION

This application claims the benefit of Provisional Application No. 63/698,681, filed Sep. 25, 2024, which is incorporated herein by reference in its entirety for all purposes.

### TECHNICAL FIELD

Instances of the present disclosure relate to wearable devices with analyte- and pH-sensing technology.

### BACKGROUND

Analyte concentrations can help physicians determine appropriate treatment for patients with heart failure, chronic kidney disease, among other ailments.

### SUMMARY

In Example 1, a method includes detecting a chemical indicator of a wearable device in a digital image; detecting a first color reference in the digital image; and determining a corrected color of the chemical indicator based, at least in part, on the first color reference.

In Example 2, the method of Example 1, further including: estimating an analyte concentration or a pH level based, at least in part, on the corrected color.

In Example 3, the method of Example 1, further including: converting the corrected color to a first color from a color coordinate system.

In Example 4, the method of Example 3, further including: estimating an analyte concentration or a pH level based, at least in part, on the first color from the color coordinate system.

In Example 5, the method of Examples 3 or 4, wherein the color coordinate system is a standardized color space.

In Example 6, the method of Examples 4 or 5, wherein the estimating the analyte concentration is based on comparing the first color to colors in a database of analyte concentrations.

In Example 7, the method of any of Examples 1-6, further including: detecting a second color reference in the digital image; and determining the corrected color of the chemical indicator based, at least in part, on the second color reference.

In Example 8, the method of any of Examples 1-7, wherein the detecting the chemical indicator includes detecting an array of chemical indicators on the wearable chemical sensor.

In Example 9, the method of Example 8, wherein the detecting the array of chemical indicators includes identifying a background portion of the wearable device and generating a digital mask over the background portion.

In Example 10, the method of Example 8 or 9, further including: determining an average color of the array of chemical indicators.

In Example 11, the method of any of Examples 1-10, wherein the detecting the chemical indicator includes detecting, in the digital image, a fiducial or pattern on the wearable device.

In Example 12, the method of any of Examples 1-11, further including: detecting a second chemical indicator of the wearable device in the digital image; determining a second corrected color of the second chemical indicator based, at least in part, on the first color reference; and determining a second analyte concentration based, at least in part, on the second corrected color.

In Example 13, a computer program product comprising instructions to cause one or more processors to carry out the steps of the method of Examples 1-12.

In Example 14, a computer-readable medium having stored thereon the computer program product of Example 13.

In Example 15, a mobile device comprising the computer-readable medium of Example 14.

In Example 16, a system includes a mobile computing device comprising an image sensor. The mobile computing device is programmed to: detect a chemical indicator of a wearable device in a digital image, detect a first color reference in the digital image, and determine a corrected color of the chemical indicator based, at least in part, on the first color reference.

In Example 17, the system of Example 16, wherein the mobile computing device is further programmed to: estimate an analyte concentration or a pH level based, at least in part, on the corrected color.

In Example 18, the system of Example 17, wherein the estimate is based on a comparison of the corrected color to colors in a database of analyte concentrations.

In Example 19, the system of claim 16, wherein the mobile computing device is further programmed to: convert the corrected color to a first color from a color coordinate system.

In Example 20, the system of Example 19, wherein the mobile computing device is further programmed to: estimate an analyte concentration or a pH level based, at least in part, on the first color from the color coordinate system.

In Example 21, the system of Example 20, wherein the color coordinate system is a standardized color space.

In Example 22, the system of Example 20, wherein the estimate is based on a comparison of the first color to colors in a database of analyte concentrations.

In Example 23, the system of Example 16, wherein the mobile computing device is further programmed to: detect a second color reference in the digital image, and determine the corrected color of the chemical indicator based, at least in part, on the second color reference.

In Example 24, the system of Example 16, wherein the mobile computing device is further programmed to: detect an array of chemical indicators, which includes the chemical indicator; identify a background portion of the wearable device; and generate a digital mask over the background portion.

In Example 25, the system of Example 16, wherein the mobile computing device is further programmed to: determine an average color and estimate an analyte concentration or a pH level based, at least in part, on the average color.

In Example 26, the system of Example 16, further including the wearable device which includes: needles sized for access to interstitial fluid, and the chemical indicator positioned within the needles.

In Example 27, the system of Example 26, wherein the chemical indicator includes a material that changes color to indicate the analyte concentration or the pH level.

In Example 28, the system of Example 26, wherein the wearable device includes the first color reference.

In Example 29, a method includes: detecting a chemical indicator of a wearable device in a digital image; detecting a first color reference in the digital image; and determining a corrected color of the chemical indicator based, at least in part, on the first color reference.

In Example 30, the method of Example 29, further including: estimating an analyte concentration or a pH level based, at least in part, on the corrected color.

In Example 31, the method of Example 30, adjusting an estimate of the analyte concentration based, at least in part, of an estimate of the pH level.

In Example 32, the method of Example 30, wherein the estimating the analyte concentration or the pH level is based on comparing the corrected color to colors in a database of analyte concentrations.

In Example 33, the method of Example 29, further including: converting the corrected color to a first color from a color coordinate system.

In Example 34, the method of Example 33, further including: estimating an analyte concentration or a pH level based, at least in part, on the first color from the color coordinate system.

In Example 35, the method of Example 29, further including: determining an average color and estimating an analyte concentration or a pH level based, at least in part, on the average color.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIGS. 2-4 show side views of various portions of a chemical sensing device, in accordance with certain instances of the present disclosure.

FIGS. 5-7 show top views of different arrangements of a chemical sensing device, in accordance with certain instances of the present disclosure.

FIGS. 8-11 show diagrams of methods for use with the chemical sensing system, in accordance with certain instances of the present disclosure.

FIG. 12 shows a block diagram of a computing device such as a mobile computing device used in connection with or to carry out methods and functions described herein, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Analyte concentration measurements and pH levels can help physicians determine treatment for patients with heart failure and chronic kidney disease, among other ailments. However, determining a patient's analyte concentrations can require drawing multiple blood samples from a patient at a clinic and processing the blood samples at a laboratory. To help reduce the need for clinic visits and laboratories, a patient can be prescribed a wearable device such as a patch that includes analyte sensing technology.

Certain instances of the present disclosure are directed to systems, methods, and devices that estimate a person's analyte concentrations and/or PH levels and that utilize wearable devices.

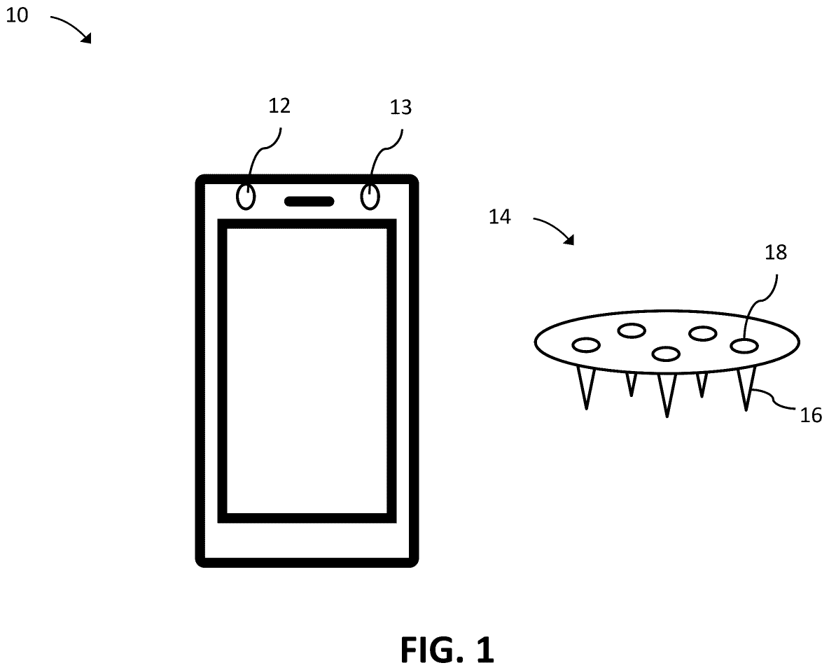

FIG. 1 shows a chemical sensing system 10 with schematic representations of a device (e.g., a mobile computing device) with an image sensor 12 and a chemical sensing device 14.

The image sensor 12 (e.g., a charge coupled device, a complementary metal oxide semiconductor, or other devices that can capture an image) can be part of a device such as a camera, smart phone, tablet, dedicated device, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a smart phone, the smart phone can store or otherwise access a program (e.g., a phone application) that processes an image (of the chemical sensing device 14) taken by the image sensor 12 and determines estimates of one or more analyte concentrations and/or pH levels of the patient. In other instances, the image sensor 12 is part of a dedicated readout device or part of a camera. The system 10 can include one or more light sources 13 (e.g., light-emitting diode(s)), which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes a mechanism (e.g., needles 16) for accessing a patient's interstitial fluid. For example, multiple needles 16 (e.g., microneedles) can be sized to access a patient's interstitial fluid. The patch 14 can also include multiple chemical indicators 18, each of which changes optical properties (e.g., fluorimetric properties, colorimetric properties) with changes in concentration of a certain analyte in the interstitial fluid or pH level of the interstitial fluid. As described in more detail herein, the image sensor 12 can be used to capture an image (e.g., a digital image) of the chemical indicators 18, and the image can be processed and analyzed to determine respective concentrations of targeted analytes or pH levels. In certain instances, the patch 14 includes one type of chemical indicator 18 (e.g., to help determine concentration of one type of analyte), but in other instances the patch 14 includes multiple types of chemical indicators.

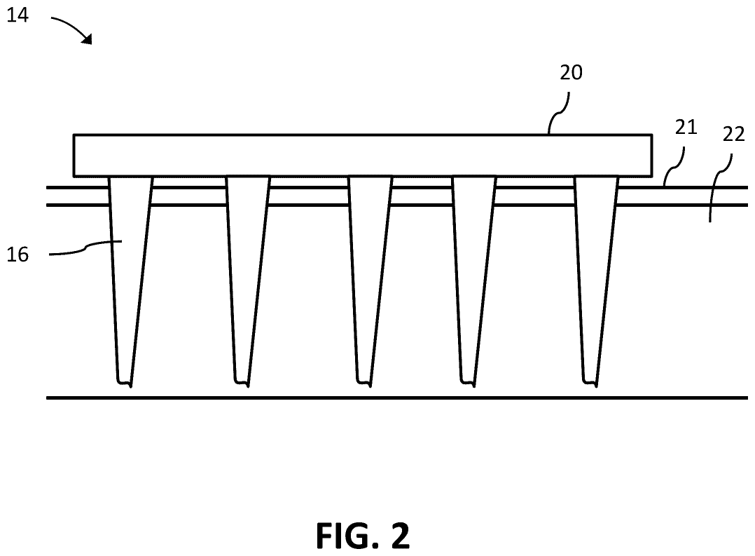

FIG. 2 shows a schematic side view of the patch 14 being worn by a patient. The patch 14 can be coupled to the patient's skin 21 such that the needles 16 pierce through the outer layer of skin 21 and extend into the patient's interstitial fluid space 22. The needles 16 can have openings that are exposed to the patient's interstitial fluid (and therefore analytes within the patient's interstitial fluid).

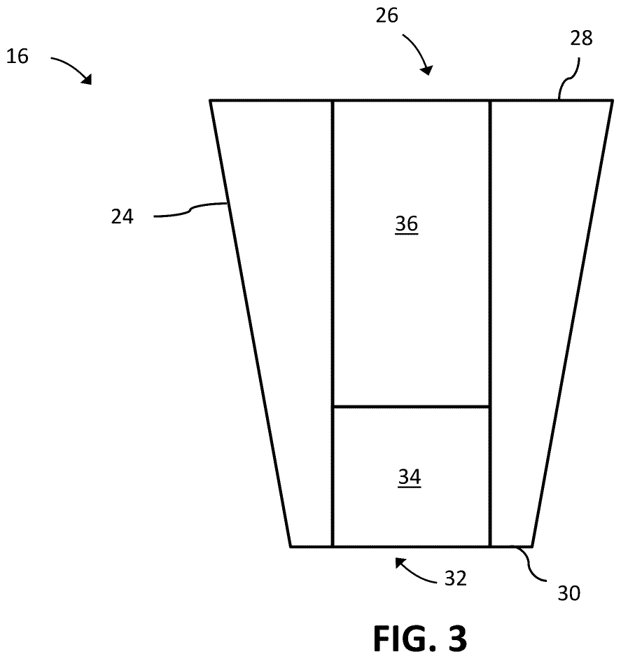

FIG. 3 shows a schematic side view of one of the needles 16 of the patch 14. In certain instances, the needles 16 are hollow needles such that each needle 16 includes an outer needle structure 24 that surrounds an opening 26 (e.g., a central thru-hole within the needle 16). The opening 26 can extend from a proximal end 28 of the needle 16 to a distal end 30 of the needle 16. An aperture 32 is located at or near the distal end 30 of the needle 16 such that the opening 26 is exposed to interstitial fluid.

Also at or near the distal end 30 of the needle 16 is a membrane 34 (e.g., a diffusion membrane) that is positioned within the needle 16. The membrane 34 protects tissue from direct interaction or exposure to a chemical indicator 36 that is also positioned within the needle 16. The membrane 34 can be formed from a permeable material, such as an ion permeable polymeric matrix material. In some instances, the membrane 34 can be permeable to protons (for pH levels), sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 34 includes an active agent disposed therein including, but not limited to, anti-inflammatory agents, angiogenic agents, and the like.

The particular type (e.g., type of ion selectivity) and length of membrane can vary by needle 16. For example, one set of needles 16 can include a membrane 34 that is permeable to sodium ions, while another set of needles 16 includes a membrane 34 that is permeable to potassium ions, and so on. In other examples, the membrane 34 is agnostic to a particular type of ion. The membrane 34 is positioned such that analytes must pass through the membrane 34 before reaching the chemical indicator 36. The membrane 34 material used will affect how fast an analyte travels between interstitial fluid and the chemical indicator 36.

The chemical indicator 36 comprises a material that changes properties (e.g., optical properties such as color) with changes in concentration of a given analyte or with changes in pH levels. In certain instances, the color of the chemical indicator 36 comprises the sum of the absorption, transmission, reflectance, and fluorescence properties of the chemical indicator material. Put another way, the chemical indicator 36 can comprise a material that changes optical properties with changes in concentration of a given analyte (or pH level)—and such optical properties can be measured by analyzing an image of the chemical indicator 36.

The particular material of the chemical indicator 36 can vary by needle 16. For example, one set of needles 16 can include a chemical indicator 36 that responds to changes in sodium concentration, while another set of needles 16 includes a chemical indicator 36 that responds to changes in potassium concentration, while another set of needles 16 includes a chemical sensor that responds to pH levels, and so on. In certain instances, the chemical indicator 36 has a minimum thickness or height along a longitudinal axis of a needle of 0.15-0.60 mm (e.g., 0.50-0.60 mm). In certain instances, the chemical indicator 36 comprises a slurry or a film.

In certain instances, the chemical indicator 36 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 36 can include components (or response elements) that are configured for a colorimetric response, a photoluminescent response, or another optical sensing modality. For example, the chemical indicator 36 can include an element that changes color based on binding with or otherwise complexing with a specific chemical analyte. In some instances, the chemical indicator 36 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 36 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

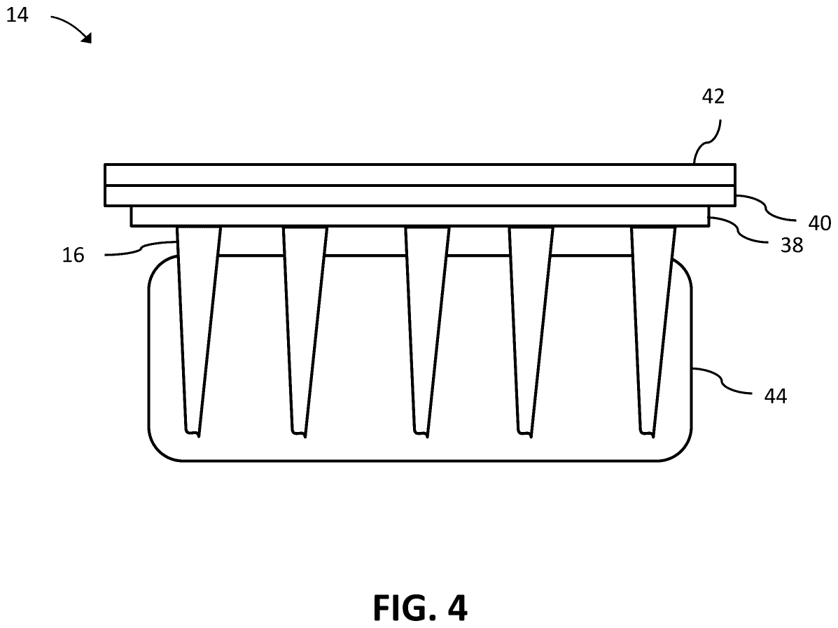

FIG. 4 shows a schematic side view of the patch 14 before being coupled to a patient (e.g., after manufacturing but before application to the patient). The needles 16 are coupled to and extend from a substrate 38. The substrate 38 can include respective openings or windows that couple to the openings 26 of the needles 16. As described in more detail below, the substrate 38 can include or be coupled to various color reference markers.

The patch 14 can also include a moisture barrier 40 that is coupled to the substrate 38. The moisture barrier 40 can include or form a window (e.g., a transparent window) such that optical properties (e.g., color) of the chemical indicators 36 (and color reference markers) can be viewed/observed from above the patch 14. The moisture barrier 40 can provide a vapor barrier such that the chemical indicators 36 (or components thereof) and analytes cannot pass through the moisture barrier 40. Put another way, the moisture barrier 40 can provide a seal that helps prevent liquid from leaking from the patch 14 such that the chemical indicators 36 remain positioned within the needles 16. The substrate 38 and the moisture barrier 40 can be flexible such that the patch 14 can be applied to curved parts of the patients and maintain contact when the patient moves.

The patch 14 can include a protective film 42 and a seal 44. The protective film 42 can be removably coupled to the moisture barrier 40 such that the protective film 42 can be removed right before or even after the patch 14 is applied to the patient. The seal 44 can be removably coupled to the needles 16 such that the needles 16 are not exposed during transit/shipping of the patch.

### Patches

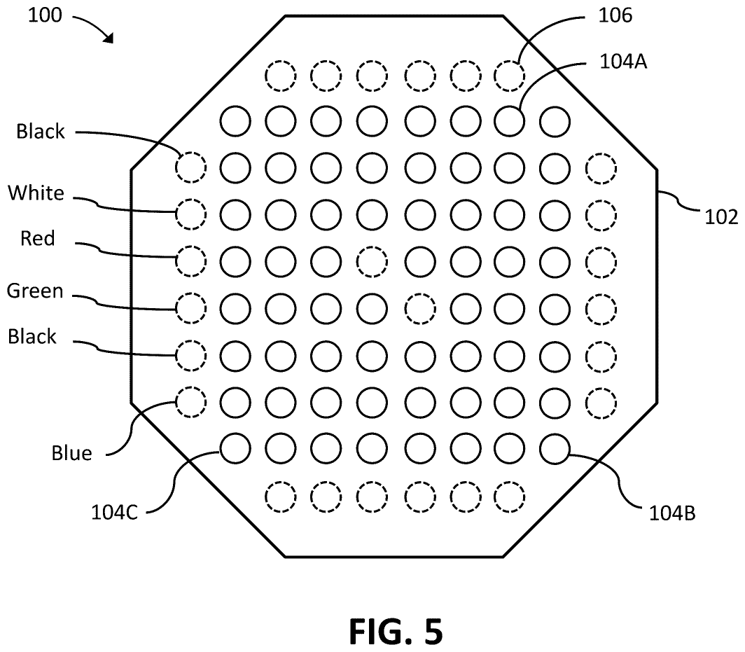

FIGS. 5-7 show top views of different arrangements of a wearable device such as a patch. Each of the arrangements can be used in connection with the needles, membranes, chemical indicators, and layers, etc., described above with respect to the patch 14. The views shown in FIGS. 5-7 are the types of views of a patch that an image sensor would capture in a digital image while the patch is coupled to the patient. The digital image can capture the colors of the chemical indicators (and color references) such that the colors can be analyzed to determine concentrations of one or more analytes of the patient and/or pH levels. The patch designs and features thereof in FIGS. 5-7 are not mutually exclusive and can be combined with each other to create other patch designs.

FIG. 5 shows a patch 100, which includes a window 102 through which various components of the patch 100 can be viewed. In particular, the window 102 allows chemical indicators 104A-C of the patch 100 to be viewed. The chemical indicators 104A-C can be positioned in needles (e.g., hollow needles) such as the needles described above with respect to FIGS. 1-4. As such, each chemical indicator 104A-C can be associated with its own needle.

A first set of needles can include a first type of chemical indicator 104A such as a chemical indicator that changes in color with changes in concentration of a first analyte (e.g., sodium) or pH levels. A second set of needles can include a second type of chemical indicator 104B such as a chemical indicator that changes in color with changes in concentration of a second analyte (e.g., potassium). A third set of needles can include a third type of chemical indicator 104C such as a chemical indicator that changes in color with changes in concentration of a third analyte (e.g., glucose). The respective colors of the chemical indicators can be used to estimate the respective concentrations of analytes in a patient's interstitial fluid. In some instances, certain needles within a set could be used for the same target analyte, but the chemical indicator formulation could vary to focus on a subrange of concentrations or environmental temperature.

In certain instances, each of the first type of chemical indicators 104A are positioned near or next to each other, each of the second type of chemical indicators 104B are positioned near or next to each other, and so on. The overall number of chemical indicators (and therefore the number of needles) and the number of different sets of types of chemical indicators on a given patch can be fewer or greater than that shown in FIG. 5. For example, the patch 100 could include a single type of chemical indicator selected for a single type of analyte. The relative positions of the chemical indicators 104A-C can vary from that shown in FIG. 5, and the specific shape of the chemical indicators 104A-C (as seen from a top view) can vary from the circular shapes shown in FIG. 5. In certain instances, the patch 100 includes a 5×5 matrix of chemical indicators.

The patch 100 can also include color references 106. The color references 106 are shown in dotted lines in FIG. 5. The color references 106 can help with calibrating, correcting, and/or processing the digital image of the chemical indicators 104A-C such that an accurate estimate of the color of the chemical indicators 104A-C can be determined. For example, because the color of the color references 106 is known, the color of the chemical indicators 104A-C can be more accurately estimated as the patch 100 is positioned in different lighting (e.g., in direct sunlight, in a shadow, partially shaded, and the like). As such, the color references 106 can act as a color index or reference point for correcting for changes in color caused by ambient light.

In certain instances, some of the color references 106 are black, others white, others red, others green, others blue. In some instances, the color references 106 are only black or white or gray (e.g., 50% reflection). Although most of the color references 106 in FIG. 5 are shown around a perimeter of the patch (e.g., with the chemical indicators 104A-C positioned within the perimeter), other positions and arrangements of the color references 106 can be utilized in the patch 100. The overall number and the specific shape of the color references 106 (as seen from a top view) can vary from the circular or dot shape shown in FIG. 5. For example, the color references 106 could be rectangular shaped.

In certain instances, the patch 100 does not include any active electronics (e.g., does not include computing components or electronic sensors). In such instances, the patch 100 does not require batteries or another power source to function as designed.

The patch 100 can include one or more fiducials to provide one or more reference points that can be used to determine the orientation of the patch 100 in a digital image. In some examples, the fiducials are simply points or edges of the patch 100, one or more color references, or one or more chemical indicators. In other examples, the top surface of the patch 100 includes a mark or surface feature that acts as a fiducial. Additionally or alternatively, in some examples, the patch 100 includes a feature that causes the patch 100 to be asymmetric such that the orientation of the patch 100 can be determined in a digital image.

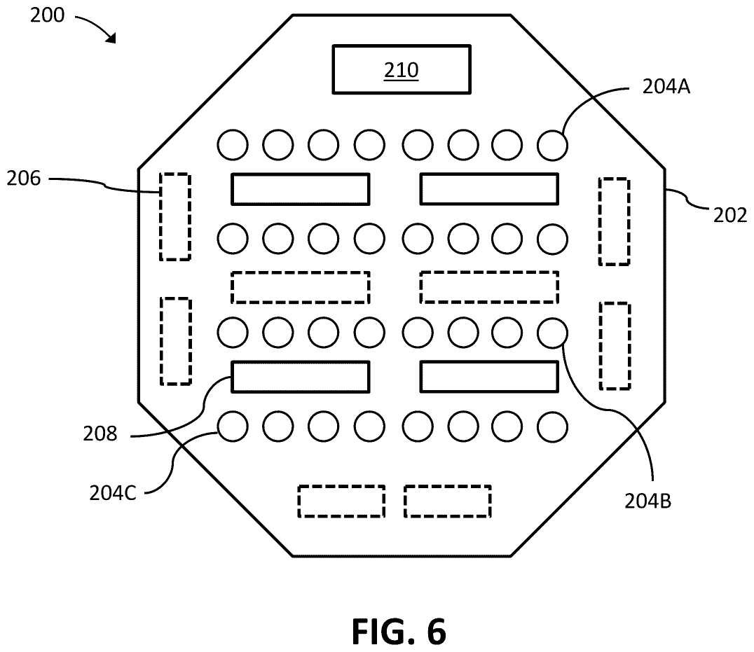

FIG. 6 shows a patch 200, which includes a window 202 through which various components of the patch 200 can be viewed. In particular, the window 202 allows chemical indicators 204A-C of the patch 200 to be viewed. The chemical indicators 204A-C can be positioned in needles (e.g., hollow needles) such as the needles described above with respect to FIGS. 1-4. As such, each chemical indicator 204A-C can be associated with its own needle.

A first set of needles can include a first type of chemical indicator 204A such as a chemical indicator that changes in color with changes in concentration of a first analyte (e.g., sodium) or pH levels. A second set of needles can include a second type of chemical indicator 204B such as a chemical indicator that changes in color with changes in concentration of a second analyte (e.g., potassium). A third set of needles can include a third type of chemical indicator 204C such as a chemical indicator that changes in color with changes in concentration of a third analyte (e.g., glucose). The respective colors of the chemical indicators can be used to estimate the respective concentrations of analytes in a patient's interstitial fluid.

In certain instances, each of the first type of chemical indicators 204A are positioned near or next to each other, each of the second type of chemical indicators 204B are positioned near or next to each other, and so on. The overall number of chemical indicators (and therefore the number of needles) and the number of different sets of types of chemical indicators on a given patch can be fewer or greater than that shown in FIG. 6. Further, the relative positions of the chemical indicators 204A-C can vary from that shown in FIG. 6. Further yet, the specific shape of the chemical indicators 204A-C (as seen from a top view) can vary from the circular shape shown in FIG. 6.

The patch 200 can also include color references 206. The color references 206 are shown in dotted lines in FIG. 6. In certain instances, some of the color references 206 are black, others white, others red, others green, others blue. The positions and arrangements of the color references 206 can vary from that shown in FIG. 6. The overall number and the specific shape of the color references 206 (as seen from a top view) can vary from the rectangular shape shown in FIG. 6.

The patch 200 can include one or more temperature sensors 208. The temperature sensors 208 can include a material (e.g., a liquid crystal material) that changes color with changes to temperature. Because the chemical indicators 204A-C may be sensitive to temperature, the color of the temperature sensors 208 can be used for calibrating, correcting, and/or processing the digital image of the chemical indicators 204A-C such that an accurate estimate of the color of the chemical indicators 204A-C can be determined.

The patch 200 can also include a bar code 210 (e.g., a one-dimensional bar code or a two-dimensional bar code). The bar code 210 can assist with identifying the patient, the model of the patch 200, the serial number of the patch 300, the arrangement of the patch 200, and/or the orientation 200 of the patch 200.

In certain instances, the patch 200 does not include any active electronics (e.g., does not include computing components or electronic sensors). In such instances, the patch 200 does not require batteries or another power source to function as designed.

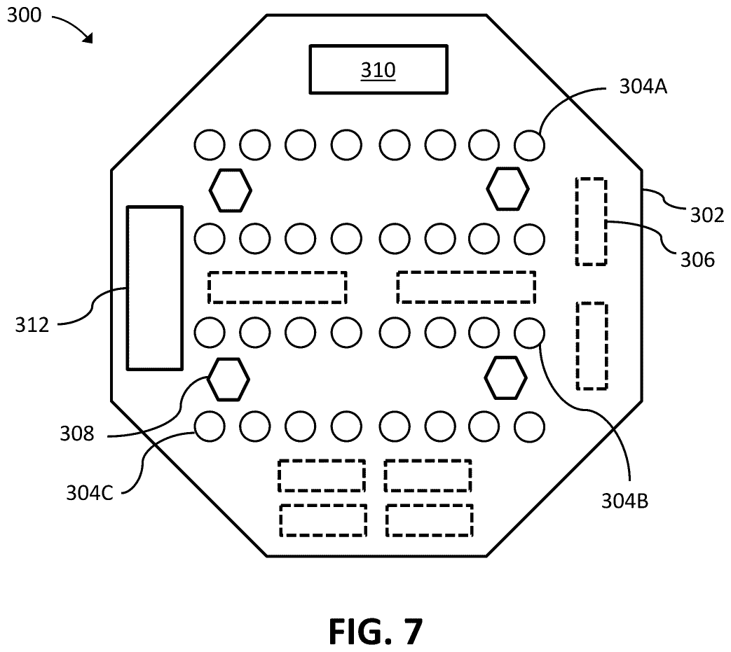

FIG. 7 shows a patch 300, which includes a window 302 through which various components of the patch 300 can be viewed. In particular, the window 302 allows chemical indicators 304A-C of the patch 300 to be viewed. The chemical indicators 304A-C can be positioned in needles (e.g., hollow needles) such as the needles described above with respect to FIGS. 1-4. As such, each chemical indicator 304A-C can be associated with its own needle.

A first set of needles can include a first type of chemical indicator 304A such as a chemical indicator that changes in color with changes in concentration of a first analyte (e.g., sodium) or PH levels. A second set of needles can include a second type of chemical indicator 304B such as a chemical indicator that changes in color with changes in concentration of a second analyte (e.g., potassium). A third set of needles can include a third type of chemical indicator 304C such as a chemical indicator that changes in color with changes in concentration of a third analyte (e.g., glucose). The respective colors of the chemical indicators can be used to estimate the respective concentrations of analytes in a patient's interstitial fluid.

In certain instances, each of the first type of chemical indicators 304A are positioned near or next to each other, each of the second type of chemical indicators 304B are positioned near or next to each other, and so on. The overall number of chemical indicators (and therefore the number of needles) and the number of different sets of types of chemical indicators on a given patch can be fewer or greater than that shown in FIG. 7. Further, the relative positions of the chemical indicators 304A-C can vary from that shown in FIG. 7. Further yet, the specific shape of the chemical indicators 304A-C (as seen from a top view) can vary from the circular shape shown in FIG. 7.

The patch 300 can also include color references 306. The color references 306 are shown in dotted lines in FIG. 7. In certain instances, some of the color references 306 are black, others white, others red, others green, others blue. The positions and arrangements of the color references 306 can vary from that shown in FIG. 7. The overall number and the specific shape of the color references 306 (as seen from a top view) can vary from the rectangular shape shown in FIG. 7.

The patch 300 can include one or more temperature sensors 308. The temperature sensors 308 can include sensors such as thermistors, thermocouples, or semiconductor junctions. Because the chemical indicators 304A-C may be sensitive to temperature, the temperature sensors 308 can be used for calibrating, correcting, and/or processing the digital image of the chemical indicators 304A-C such that an accurate estimate of the color of the chemical indicators 304A-C can be determined.

The patch 300 can also include a bar code 310. The bar code 310 can assist with identifying the patient, the model of the patch 300, the serial number of the patch 300, the arrangement of the patch 300, and/or the orientation 300 of the patch 300.

The patch 300 can also include a radio frequency identification (RFID) chip 312. In certain instances, the RFID chip 312 includes the temperature sensors 308. Further, the RFID chip 312 can store information such as identifying the patient, the model of the patch 300, the serial number of the patch 300, and the arrangement of the patch 300, etc. The RFID chip 312 can be designed to pair with certain reading devices that can access the information wirelessly.

In certain instances, the patch 300 does not include any active electronics (e.g., does not include computing components or electronic sensors). In such instances, the patch 300 does not require batteries or another power source to function as designed.

The various features of the patches 100, 200, and 300 described herein can be combined with each other to create different patch designs than those specifically shown in FIGS. 5-7.

### Methods

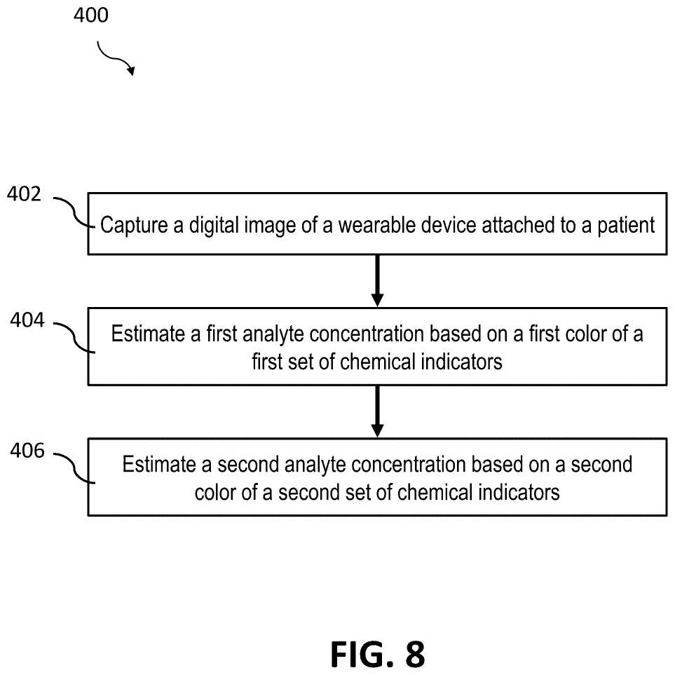

FIG. 8 shows a block diagram of a method 400 for estimating one or more analyte concentrations and/or pH levels. The method 400 includes capturing a digital image of a wearable device attached to a patient (block 402). The method 400 further includes estimating a first analyte concentration or pH level based on a first color of the first set of chemical indicators (block 404). The method 400 further includes estimating a second analyte concentration or pH level based on a second color of the second set of chemical indicators (block 406). In certain instances, estimating the analyte concentrations and pH levels involves calculating an analyte concentration for multiple chemical indicators and then applying a mathematical operation (e.g., averaging, voting) to determine the respective analyte concentrations or PH levels. The analyte concentration estimations or pH levels can be further based on corrections that are determined using color reference sections of the wearable device (e.g., a patch).

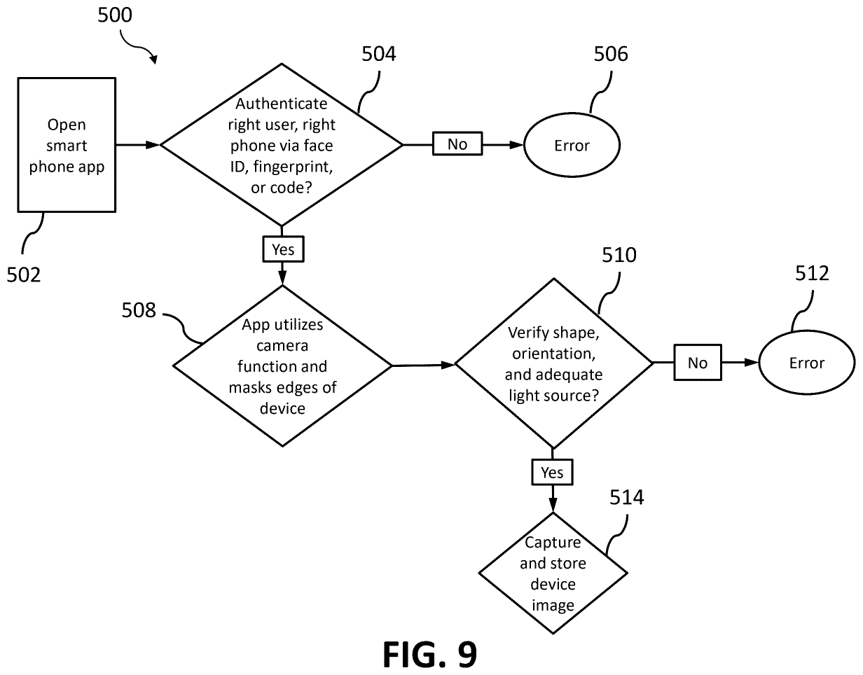

FIG. 9 shows a flowchart of a method 500 for capturing an image of a wearable device using an application on a smart phone. After opening the application (block 502) or even before the application is opened, the smart phone runs an authentication process to confirm that the correct patient is using the smart phone or application (block 504). The authentication process can involve checking items such as a fingerprint, facial recognition, a code or password, and/or the like. If the patient cannot be authenticated, the application determines that an error has occurred (block 506). Once authentication is confirmed, the application can access one or more cameras of the smart phone (block 508). In certain instances, the camera function of the smart phone can be used to determine and mask the edges of the wearable device. Next, the application can verify various aspects of the wearable device (block 510). This can involve verifying the shape of the wearable device, the orientation of the wearable device, and adequate light source. If certain aspects of the wearable device cannot be verified, the application determines that an error has occurred (block 512). Once the wearable device is verified, the one or more cameras can capture a digital image of the wearable device and store the digital image to local memory of the smart phone (block 514).

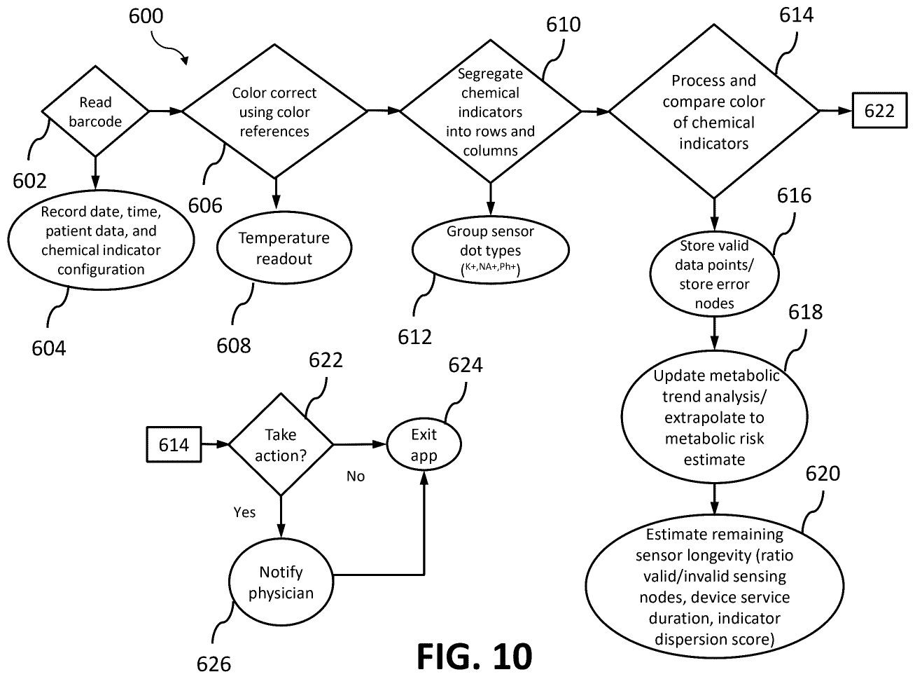

FIG. 10 shows a flowchart of a method 600 for determining estimated analyte concentrations and/or pH levels based on an image captured by an image sensor. The method 600 can include reading a barcode (or other identifying indicator) from the digital image or otherwise accessing identifying information about a wearable device such as via an RFID chip (block 602). In response, the method 600 can include recording information (block 604) such as the date, time, patient data, and wearable data (e.g., arrangement of chemical indicators, type of chemical indicators).

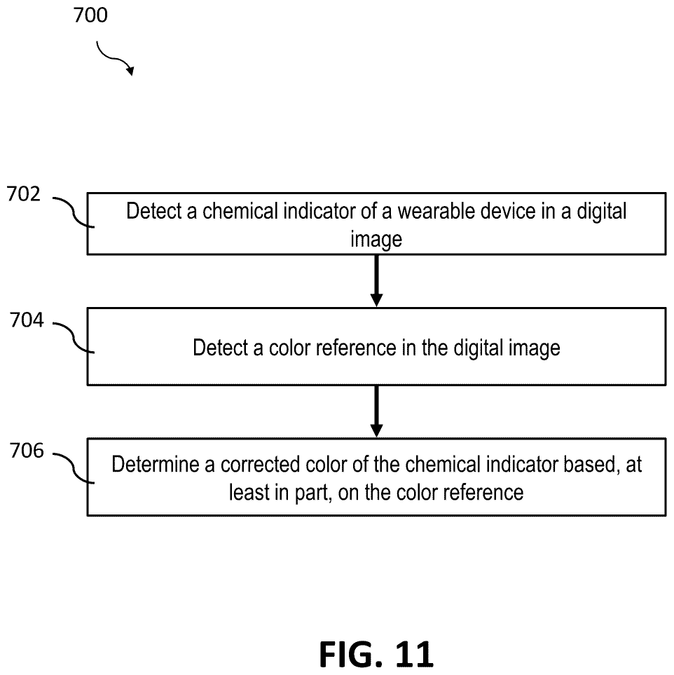

The method 600 further includes correcting colors of the chemical indicators from the digital image using color references from the digital image (block 606). Additional details of how color can be corrected is described herein with respect to FIG. 11.

Further, the method 600 can include determining temperature (block 608) based on information in the digital image or via another process. If the wearable device includes multiple temperature sensors, the overall temperature can be determined by averaging the temperature measurements.

The chemical indicators in the digital image can be segregated into separate sets, with each set grouping together chemical indicators that are the same type (blocks 610 and 612). In certain instances, the relative positions of the chemical indicators are used to determine which type each chemical indicator is.

Each set or grouping of chemical indicators from the digital image are processed and their respective colors are compared to a table, library, mapping, index, etc. that associates a given color of chemical indicator to a given concentration level (block 614). As previously noted, the estimated concentration level or pH level can be based on averaging the individual concentration levels associated with each chemical indicator or by a voting mechanism. As part of processing the chemical indicators, certain individual chemical indicators can be determined to be valid or invalid (e.g., associated with an error) (block 616). For example, a chemical indicator may be determined to be invalid if it is determined that the needle with the chemical indicator has detached from the patient or otherwise has an issue that affects the color of the chemical indicator.

Further, in certain instances, the method 600 can include using the estimated concentration level(s) to update a database of historical concentration levels and analyze the concentration levels to determine trends and potential health risks (block 618). Further, in certain instances, the method 600 can include periodically estimating the remaining longevity or life of the wearable device (block 620). This can include analyzing information such as the ratio of valid to invalid chemical indicators, the service duration of the wearable device, the dispersion or spread of colors of the chemical indicators.

In certain instances, the method 600 includes determining whether the estimated analyte concentrations warrant treatment or action (block 622). If not, the method 600 can conclude (block 624). But if action is warranted, a notification (e.g., electronic message) can be sent to the patient's physician (block 626) along with the underlying data that caused the notification.

In certain instances, the method 600 is carried out by an application stored on and operated by a smart phone. In other instances, some or all steps of the method 600 can be carried out by a server or other computing system besides a smart phone that can access digital images of a wearable device and be programmed to determine estimated analyte concentration levels based on colors of chemical indicators shown in the digital image.

FIG. 11 shows a block diagram of a method 700 for estimating one or more analyte concentrations and/or PH levels.

The method 700 includes detecting one or more chemical indicators of a wearable device (e.g., a patch) in a digital image (block 702 in FIG. 11). The method 700 further includes detecting one or more color references in the digital image (block 704 in FIG. 11). The process of detecting chemical indicators and/or one or more color references in a digital image can include multiple feature-recognition techniques.

One technique involves detecting a pattern such as the pattern or array of chemical indicators, color references, and/or surface features on the wearable device. In certain instances, in connection with detecting the pattern, the digital image (or the wearable device as-displayed in the digital image) is oriented to a standard orientation (e.g., orientation, size, straightness/flatness, aspect ratio). This can include detecting one or more fiducials on the patch to determine a reference point and then orienting the digital image to a standard orientation. Detecting fiducials can include detecting corners (e.g., top-left corner and bottom-right corner) of the patch or array of chemical indicators. In certain instances, a barcode or other identification marker on the wearable device is detected and used to indicate what pattern or array of chemical indicators, types of chemical indicators, etc., is associated with the given wearable device in the digital image. The barcode (or another type of identification marker) could be used as the fiducial or reference point.

Another technique for detecting chemical indicators and/or color references of the wearable device involves identifying a background portion of the wearable device in the digital image and then generating a digital mask over the identified background portion. The background portion of the wearable device (e.g., the portion that does not include the chemical indicators or color references) can be colored such that the chemical indicators and color references can be identified in a digital image. In certain instances, the background portion is identified based, at least in part, on RGB value ranges in the digital images. In other instances, the wearable-device-type is identified using the bar code. In such instances, the background portion of the digital image is masked according to a model specific digital mask, leaving relevant chemical indicators in relief.

In addition to identifying and masking the background portion, certain chemical indicators (or portions thereof) that are within (or not within) certain color ranges (e.g., RGB value ranges) can be identified and masked. For example, some chemical indicators may malfunction and therefore should be ignored for purposes of estimating analyte concentrations or pH levels. In some instances, after a given chemical indicator has been ignored a certain number of percentage of times, that chemical indicator can be flagged as unreliable and ignored in any future digital image. As another example, some chemical indicators (or portions thereof) in the digital image may be too bright or too dark and therefore should be ignored for purposes of estimating analyte concentrations or pH levels. In some instances, identifying the background portion involves first identifying chemical indicators and color references (e.g., based on color ranges) and then marking any portion not identified as a usable section of a chemical indictor or color reference as being part of the background portion.

As noted herein, one or more detected color references can be used to correct color of the detected chemical indicators. The color references can be used as a reference to compensate for different lighting conditions. As such, a corrected color of the chemical indicator can be determined based, at least in part, on one or more color references (block 706 in FIG. 11). As noted herein, the color references can be part of the wearable device itself such that the color references and the chemical indicators are exposed to similar lighting conditions.

In certain instances, the color references are used to determine whether a digital image is usable. For example, if a minimum number of color references cannot be identified (e.g., because of the angle of the digital image of the wearable device), the digital image can be ignored and not used to estimate analyte concentrations, etc. As another example, if a minimum usable area of color references cannot be identified, the digital image can be ignored and not used to estimate analyte concentrations, etc. As another example, if the wearable device is partially exposed to direct sunlight and partially shaded, the color references can be used to determine that a digital image is not usable.

Regardless of which approach is used to determine whether a digital image is usable, an alert or message can be generated and displayed on a user interface to instruct the user to take another digital image of the wearable device. In some instances, the alert or message can provide specific instructions on how to correct the error from the previous digital image (e.g., take a digital image at a less of an angle, take a digital image when the wearable device is not in direct sunlight, use a light source to illuminate the wearable device while taking a digital image).

The corrected color can then be used to estimate an analyte concentration or a pH level based, at least in part, on the corrected color. In certain instances, before estimating an analyte concentration or a pH level, the corrected color is converted to a color from a color coordinate system. Example color coordinate systems can include systems referred to as a standardized color space or color model such as those maintained by the International Commission on Illumination (CIE). These standardized color spaces can include color spaces referred to as XYZ, LAB, HSV, and the like. In instances where the corrected color is converted, the color from the color coordinate system is used to estimate an analyte concentration or a pH level.

In certain instances, estimated analyte concentrations can be corrected based on estimated pH levels. For example, potassium concentration readings and sodium concentration readings can be affected by pH levels. As such, in some instances, the pH levels estimated based on one set of chemical indicators of the wearable device can be used to calibrate or otherwise correct concentration readings of analytes estimated based on another set of chemical indicators of the wearable device. Other corrections such as temperature-based corrections (as described herein) can be applied as part of estimating analyte concentrations and/or PH levels.

Regardless of how or whether the color of the chemical indicators are corrected, converted, etc., estimating analyte concentrations and pH levels can involve various techniques.

One technique involves calculating an average color of groups of chemical indicators. For example, an average color of chemical indicators intended to sense one analyte (or pH level) can be calculated. An average color of other chemical indicators intended to sense another analyte (or pH level) can be calculated. The average color(s) can be calculated before or after correcting the color of the chemical indicators in the digital image and/or converting the color to one from a color coordinate system.

Another technique involves comparing the color (or average color) of the chemical indicators to a database, library, etc., of colors—where a given color is associated with a given analyte concentration or pH level. As such, the color (or average color) can be correlated to a specific analyte level using a database, library, etc., of color-to-analyte-concentration relationships or color-to-pH-level relationships.

In certain instances, the steps and techniques described with respect to FIG. 11 are carried out by a single device (e.g., a mobile computing device such as a smartphone or tablet, or a dedicated device at a patient's home or clinic). For example, the single device could take the digital image of the wearable device, detect one or more chemical indicators on the wearable device, detect one or more color references on the wearable device, determine a corrected color, convert the color or corrected color to a color from a color model, and estimate an analyte concentration and/or a pH level. In other instances, at least some of the steps and techniques can be carried out by a separate device such as a server. For example, the server could store the database of color-to-analyte-concentration relationships or color-to-pH-level relationships and generate the estimated concentrations and/or pH levels.

In instances where the device is a dedicated device (instead of a smartphone/tablet with an app), the device can be a scanner/reader (e.g., a housing with an image sensor, light source, circuitry) that is specifically made and adapted for taking and processing digital images of a wearable device described herein. Such a dedicated device could be designed to be placed in a hospital room, clinic, home, etc.

### Computing Device

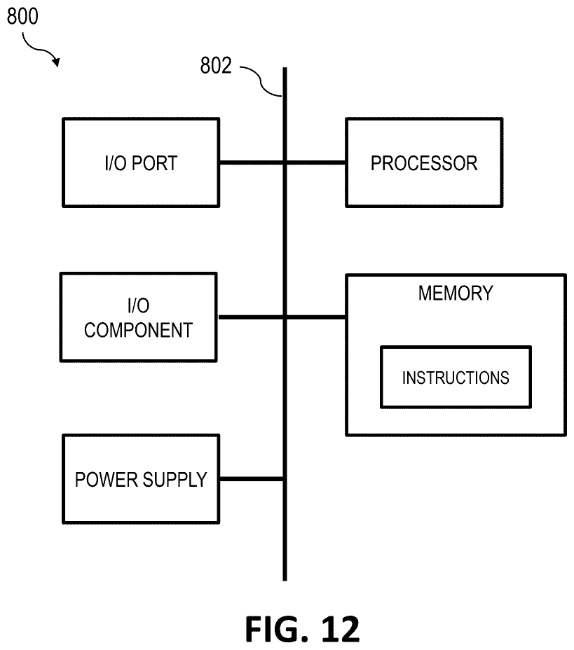

FIG. 12 is a block diagram of a computing device 800. The computing device 800 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like.

In instances, the computing device 800 includes a bus 802 that, directly and/or indirectly, couples one or more of the following devices: a processor, a memory, an input/output (I/O) port, an I/O component, and a power supply. Any number of additional components, different components, and/or combinations of components may also be included in the computing device 800.

The bus 802 represents what may be one or more busses (such as, for example, an address bus, data bus, or combination thereof). Similarly, in instances, the computing device 800 may include a number of processors, a number of memory components, a number of I/O ports, a number of I/O components, and/or a number of power supplies. Additionally, any number of these components, or combinations thereof, may be distributed and/or duplicated across a number of computing devices.

In instances, the memory includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory stores computer-executable instructions for causing the processor to implement aspects of instances of components discussed herein and/or to perform aspects of instances of methods and procedures discussed herein. The memory can comprise a non-transitory computer readable medium storing the computer-executable instructions.

The computer-executable instructions may include, for example, computer code, machine-useable instructions, and the like such as, for example, program components capable of being executed by one or more processors (e.g., microprocessors) associated with the computing device 800. Program components may be programmed using any number of different programming environments, including various languages, development kits, frameworks, and/or the like. Some or all of the functionality contemplated herein may also, or alternatively, be implemented in hardware and/or firmware.

According to some instances, for example, the instructions may be configured to be executed by the processor and, upon execution, to cause the processor to perform certain processes. In certain instances, the processor, memory, and instructions are part of a controller such as an application specific integrated circuit (ASIC), field-programmable gate array (FPGA), and/or the like. Such devices can be used to carry out the functions and steps described herein.

The I/O component may include a presentation component configured to present information to a user such as, for example, a display device, a speaker, a printing device, and/or the like, and/or an input component such as, for example, a microphone, a joystick, a satellite dish, a scanner, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device comprising an image sensor, wherein the mobile computing device is programmed to:
detect a chemical indicator of a wearable device in a digital image,
detect a first color reference in the digital image, and
determine a corrected color of the chemical indicator based, at least in part, on the first color reference.

2. The system of claim 1, wherein the mobile computing device is further programmed to: estimate an analyte concentration or a pH level based, at least in part, on the corrected color.

3. The system of claim 2, wherein the estimate is based on a comparison of the corrected color to colors in a database of analyte concentrations.

4. The system of claim 1, wherein the mobile computing device is further programmed to: convert the corrected color to a first color from a color coordinate system.

5. The system of claim 4, wherein the mobile computing device is further programmed to: estimate an analyte concentration or a pH level based, at least in part, on the first color from the color coordinate system.

6. The system of claim 5, wherein the color coordinate system is a standardized color space.

7. The system of claim 5, wherein the estimate is based on a comparison of the first color to colors in a database of analyte concentrations.

8. The system of claim 1, wherein the mobile computing device is further programmed to:
detect a second color reference in the digital image, and
determine the corrected color of the chemical indicator based, at least in part, on the second color reference.

9. The system of claim 1, wherein the mobile computing device is further programmed to: detect an array of chemical indicators, which includes the chemical indicator; identify a background portion of the wearable device; and generate a digital mask over the background portion.

10. The system of claim 1, wherein the mobile computing device is further programmed to: determine an average color and estimate an analyte concentration or a pH level based, at least in part, on the average color.

11. The system of claim 1, further comprising:
the wearable device that includes:
needles sized for access to interstitial fluid, and
the chemical indicator positioned within the needles.

12. The system of claim 11, wherein the chemical indicator includes a material that changes color to indicate the analyte concentration or the pH level.

13. The system of claim 11, wherein the wearable device includes the first color reference.

14. A method comprising:
detecting a chemical indicator of a wearable device in a digital image;
detecting a first color reference in the digital image; and
determining a corrected color of the chemical indicator based, at least in part, on the first color reference.

15. The method of claim 14, further comprising: estimating an analyte concentration or a pH level based, at least in part, on the corrected color.

16. The method of claim 15, adjusting an estimate of the analyte concentration based, at least in part, of an estimate of the pH level.

17. The method of claim 15, wherein the estimating the analyte concentration or the pH level is based on comparing the corrected color to colors in a database of analyte concentrations.

18. The method of claim 14, further comprising: converting the corrected color to a first color from a color coordinate system.

19. The method of claim 18, further comprising: estimating an analyte concentration or a pH level based, at least in part, on the first color from the color coordinate system.

20. The method of claim 14, further comprising: determining an average color and estimating an analyte concentration or a pH level based, at least in part, on the average color.
